(19) United States
(12) Patent Application Publication
(10) Pub. No.: US 2013/0122454 A1
(43) Pub. Date: May 16, 2013

(54) Impression Device and Method for Three-Dimensional Acquisition of Intraoral Structures, and Corresponding Scanning Device

(71) Applicant: Ludwig-Maximilians-Universitaet Muenchen, Muenchen (DE)

(72) Inventor: Josef Schweiger, Bergen (DE)

(73) Assignee: Ludwig-Maximilians-Universitaet Muenchen, Muenchen (DE)

(21) Appl. No.: 13/677,959

(22) Filed: Nov. 15, 2012

(30) Foreign Application Priority Data
Nov. 15, 2011 (DE) 102011055356.8

(51) Int. Cl. A61C 9/00; A61B 6/14

(52) U.S. Cl. CPC A61C 9/004; A61C 9/00; A61B 6/14; A61B 6/145
USPC 433/29; 433/40; 433/215

(57) ABSTRACT

The invention relates to an impression device and a method for three-dimensional acquisition of intraoral structures as well as a corresponding scanning device. The impression device of the invention comprises an impression sleeve, which has an open and a closed end, and at least one marker which is three-dimensional and disposed on the outside of the impression sleeve.

### FIELD OF THE INVENTION

The invention relates to an impression device and a method for three-dimensional acquisition of intraoral structures, in particular of teeth and prepared tooth stumps, as well as to a corresponding scanning device.

### RELATED PRIOR ART

In the field of taking impressions of a patient's intraoral structures, in the art a distinction is made between analogue acquisition with the aid of an impression and digital acquisition using 3D acquisition apparatus. The technique known as impression scanning is a combination of both methods, in which an analogue impression is digitized with the aid of a 3D laboratory scanner.

When taking analogue impressions of teeth and soft tissues, various impression materials may be used. In general, a distinction is made between rigid impression materials (impression plaster, zinc oxide-eugenol pastes, polymeric impression materials), reversible rigid impression materials (impression waxes, gutta percha, composite impression materials) and elastic impression materials. The latter are again divided, into irreversibly elastic impression materials (alginates, polysulphides, polyethers, silicones) and reversibly elastic impression materials such as hydrocolloids.

The analogue impression methods can be further divided into ring-supported impressions using what is known as a Kerr copper ring impression and an integrated complete impression. A variation of ring-supported impression is total impression, which breaks down into the following steps:
1. drying the prepared tooth stump;
2. isolating the tooth stump with Vaseline;
3. degreasing a copper ring that has been fitted closely to the tooth stump;
4. plastic heating of a Kerr material;
5. pushing the plastic Kerr material into the copper ring;
6. placement on the tooth stump;
7. withdrawal from the tooth stump and inspection;

Steps 1 to 7 can be carried out for several or even all tooth stumps.

8. placing all of the copper ring impressions on the tooth stumps again;

9. producing a total impression with an elastic material; and

10. producing a tooth model, whereby the copper rings are cast with a special stump material, whereupon the complete model is prepared in plaster.

When preparing an integrated overall impression, both the individual stumps and also the whole jaw are captured in a general impression procedure. If preparation of the teeth is sub-gingival, the gum line must be dilated using suitable means, for example retraction cords, so that the impression material can penetrate into these regions as well.

The prior art also discloses digital three-dimensional impression methods using intraoral 3D scanners. Herein, the term “digital impression” should not be misunderstood as an “impression” in a physical sense. Rather, this term of course refers to a 3D image that however provides similar information as a conventional physical impression, which for clarity is also referred to as “analogue impression” herein. All of the currently available commercial digital systems operate with optical light acquisition technologies. Known examples are systems which are based on triangulation methods or confocal imaging. In addition, other physical acquisition methods are in the discussion stage, for example sonography, acquisition by means of magnetic resonance tomography, computer tomography or digital volume tomography. Until now, however, no such system has been made available on the market.

In an impression scan, in a first step a conventional analogue impression is prepared which, however, is not cast in a subsequent step with plaster but is digitized with the aid of a laboratory scanner. Such laboratory scanners usually operate on an optical light basis, using laser light sectioning methods, stripe light projection or conoscopic cholography. All optical light systems have a problem, however, in that undercut regions cannot be acquired in the impression.

Analogue impression methods also suffer from the disadvantage that they also give rise to a gagging reflex when taking the impression or a feeling of helplessness in the patient. Furthermore, the unpleasant taste of the impression material is often complained about.

Particular disadvantages of digital impression-taking include the fact that sub-gingival regions of prepared teeth are not accessible for long enough to be acquired since they are often covered by the gingivae or saliva.

An impression scan will suffer from both the disadvantages of an analogue and the disadvantages of a digital impression.

U.S. Pat. No. 3,304,608 A describes a two-part impression sleeve wherein a first sleeve which is closed on one side and filled with an impression material is introduced into a second sleeve which is formed as a hollow cylinder, open at both ends and already pushed over the tooth from which an impression is to be taken, wherein the internal diameter of the second sleeve essentially corresponds to the external diameter of the first sleeve. The second sleeve has through openings at its distal end, out of which superfluous impression material can escape.

DE 10 2009 044 147 A1, moreover, describes a method for producing a digital data model for at least a part of an upper jaw, wherein at least one digital upper jaw model is produced by scanning at least a part of the upper jaw or a model of the upper jaw or at least a part of an impression of the upper jaw or the upper jaw model by means of a scanning device. The method envisages that in addition, at least one impression of a tooth or of several teeth of the upper jaw is scanned in a facebow registration of a bite fork in its position relative to a coordinate system assigned to the facebow by means of the scanning device, and a digital impression model is produced therefrom. Next, the position of the digital upper jaw data model in the coordinate system assigned to the facebow is set by comparing the digital upper jaw data model with the digital impression data model.

### SUMMARY OF THE INVENTION

The problem underlying the invention is to provide means and a method for three-dimensional acquisition of intraoral structures which on the one hand are patient-friendly and on the other hand allow precise three-dimensional acquisition of intraoral structures, in particular teeth and prepared tooth stumps.

This problem is solved by an impression device according to one of the embodiments described below. A further aspect of the invention relates to a system comprising such an impression device and an extraoral scanning device. A yet further aspect of the invention relates to a method for three-dimensional acquisition of intraoral structures as set forth below.

The impression device of one embodiment of the invention comprises an impression element which can be placed on a tooth or a tooth stump in order to take an impression of the tooth or the tooth stump. Further, the impression element comprises at least one marker, which is disposed on the impression element in a manner such that the position and the orientation of the impression element placed on the tooth or on the tooth stump can be determined by means of an intraoral scan. In one embodiment, the impression device comprises at least one referencing marker. Alternatively, in another embodiment of the invention, it can be connected to at least one referencing marker. The referencing marker is respectively arranged such that it can be scanned together with the impression in an extraoral scan.

By means of the impression device of the invention, it is possible to digitize even those regions of the oral cavity that cannot be acquired using prior art intraoral scanning. This is particularly the case with sub-gingival structures. In accordance with an embodiment of the invention, a two-step scanning procedure is provided. In a first step, the oral cavity including the impression device is scanned with the impression device placed on the tooth or the tooth stump. During this intraoral scan, the marker of the impression device is also scanned, which marker is arranged in a predetermined position relative to the impression device. The result of the intraoral scan is a first data set which contains 3D information concerning the major part of the oral cavity and the position and orientation of the impression device in the oral cavity, but not regarding the structures which are covered by the impression device. However, precisely these structures are recorded in analogue manner by means of the impression. As a result, after the intraoral scan, all of the 3D information has been recorded, but as yet it is not all in a digital form.

In a second step, the missing digital information is captured by means of an extraoral scan of the impression. The data set obtained thereby contains the 3D information which is missing in the first data set. In order to obtain a complete data set, i.e. a complete digital 3D image of the oral cavity, the two data sets have to be combined together. In the art, this is normally known as “registration”. Registration is carried out with the assistance of the referencing marker which is scanned together with the extraoral scan of the impression and for which its position with respect to the marker of the impression device and therefore with respect to the first data set is known.

As already discussed, depending on the embodiment, the referencing marker can be either a part of the impression device or can be connected with the impression device. Thus, for example it is possible for the referencing marker to be attached to an external side of the impression device in a manner such that on the one hand it can be acquired by the extraoral scan, on the other hand however, that it does not cover substantial regions of the oral cavity that should be acquired during the intraoral scan.

In another embodiment, the referencing marker is a component of an extraoral scanning device, wherein the referencing marker is again arranged on the extraoral scanning device such that, together with the impression, it can be scanned in the extraoral scanning device. In this embodiment, then, the impression element itself need not have any referencing markers which would be scanned during the extraoral scan. However, in this embodiment, the referencing marker is “connectable” with the impression device, namely via the extraoral scanning device. The term “connectable” as used in the present disclosure should be construed in this broad sense. In this embodiment, it is important that the impression element be positioned exactly in the extraoral scanning device for the extraoral scan, or at least exactly with respect to the referencing marker. The positioning means described below in more detail for spatially exact positioning of the impression element with respect to the extraoral scanning device have proved to be particularly suitable in this regard.

While embodiments in which the referencing marker is a component of the impression device have the advantage that the spatial arrangement of the referencing marker with respect to the impression device is predetermined with the greatest precision, embodiments in which the referencing marker is provided on the extraoral scanning device have the advantage that the referencing marker on the impression device can be dispensed with, whereby the impression device is technically simpler and thus can be produced more cheaply. Also, with a referencing marker which is arranged directly on the impression element, there is a risk that during the intraoral scan, the referencing marker will cover over substantial regions of the oral cavity, thus rendering them impossible to scan. This problem can be avoided with a referencing marker which is not permanently connected to the impression device, but is only “connectable” for the purposes of the extraoral scan.

A general fundamental concept of the invention is that, for registration of the two data sets, a marker and a referencing marker are provided which each have a known relationship to an impression element, which is scanned in both an intraoral scan and also in an extraoral scan, so that the known relationship of the marker or referencing marker to the impression device allows for a registration of the data set obtained during the intraoral scan with the data set obtained during the extraoral scan. Consequently, in accordance with the invention, the arrangement of the marker or the referencing marker with respect to the impression device is not limited to the examples described herein.

In one embodiment of the invention, the impression element has a sleeve to accommodate an impression material. The sleeve has an open and a closed end and the marker is disposed on the closed end.

As an example, the marker can be formed at least in part as a sphere. Furthermore, the marker may have at least one lateral flat which, depending on the embodiment, may be at least partially planar. In a particularly preferred embodiment, the marker has at least two flats which are at least in part arranged in mutually perpendicular planes. For particularly precise scan results, the marker preferably combines several of the features mentioned above. A particularly effective marker is thus formed at least in part as a sphere and comprises at least two planar flats which are at least in part arranged in mutually perpendicular planes.

In another embodiment, the marker is at least partially pyramidal in shape. The pyramidal section of the marker may be a pyramid with a triangular base, a square base or a rectangular base.

In an alternative embodiment, the marker has at least one spherical element. Thus, for example, the marker may have four hemispherical elements which are preferably arranged in a common plane at the corners of a square or a rectangle.

In a further embodiment of the invention, the marker comprises at least two spheres arranged one above the other, wherein at least one of the spheres has at least one flat. Particularly preferably, each of the spheres has at least two flats, wherein each sphere has two flats which are at least partially planar and orientated perpendicular to each other.

Preferably again, the marker is connected with the impression sleeve via a connecting element, wherein the connecting element extends from the closed end of the impression sleeve. Preferably, the connecting element has a connecting surface via which the at least one marker makes contact with the connecting element. In this manner, the connecting surface can extend parallel to the closed end of the impression sleeve.

Preferably, the connecting element has a further function whereby it comprises or forms positioning means for spatially exact positioning of the impression device in an extraoral scanning device or in a holder thereof. In this way, the impression device according to one of the above embodiments and the extraoral scanning device form a system for three-dimensional acquisition of intraoral structures according to a further aspect of the present invention. In the context of this embodiment of the invention, the positioning means are not limited in any way. In order to allow the impression device to be inserted into or removed from the scanning device easily, the positioning means may have at least one groove or tongue of a tongue-and-groove connection. Particularly preferably, an interlocking dovetail connection is formed between the extraoral scanning device or the holder and the impression device via the connecting element with the aid of the positioning means for spatially exact positioning. This has the advantage that the impression device can be linearly displaced along the axis of symmetry of the tongue-and-groove connection in order to fasten it in the extraoral scanning device.

In an advantageous embodiment, the marker (also) comprises positioning means for spatially exact positioning of the impression device in an extraoral scanning device. These means can, for example, be a guide groove, which is formed on one of the ends of the impression sleeve distal to the marker. When the impression element is inserted into an extraoral scanning device, the guide groove of the marker can engage with a guide rib of the extraoral scanning device. Advantageously, the axis of symmetry of the guide groove is orientated parallel to the axis of symmetry of the tongue-and-groove connection, if it is present.

Preferably, the means for spatially exact positioning of the impression device in the extraoral scanning device are complementary, so that whenever the impression device or the extraoral scanning device has first means for spatially exact positioning of the impression device, the other component has means that complete the first means so that by their cooperation, spatially exact positioning of the impression device in an extraoral scanning device is accomplished. An example of such cooperating means could be the already mentioned tongue-and-groove connection, wherein one of the two components (i.e. impression device or extraoral scanning device) has the groove and the other has the tongue. It is likewise conceivable that the extraoral scanning device or the holder comprises either a guide rib or the guide groove, which can be engaged with the respective other component which is formed on the end of the marker distal to the impression sleeve.

A method according to one embodiment of the invention for three-dimensional acquisition of intraoral structures, in particular teeth and prepared tooth stumps, comprises the following steps:

providing an impression device constituted as described above, wherein the impression element of the impression device is filled with an impression material;

placing the impression device on a tooth or a tooth stump in an oral cavity, such that an analogue impression of the tooth or the tooth stump is produced in the impression material;

scanning the oral cavity including the marker disposed on the impression element with an intraoral scanner and producing a first data set which comprises a digitized, three-dimensional image of the oral cavity and the marker;

removing the impression device from the oral cavity and scanning the analogue impression as well as the referencing marker and producing a second data set which comprises a digitized three-dimensional image of the analogue impression and the referencing marker.

When an extraoral scanning device is to be used to scan the analogue impression and the referencing marker, the impression device can be inserted in an extraoral scanning device in accordance with one of the embodiments described above, wherein care should be taken regarding the spatially exact arrangement of the impression device in particular with respect to the referencing marker of the scanning device.

In this respect, after the above-mentioned steps, the method of the invention comprises combining the first and second data set, calling upon a known spatial relationship between the at least one marker of the impression device and the referencing marker.

In an alternative embodiment, the marker of the impression device and the referencing marker can be dispensed with and the registration is undertaken on the basis of the recorded three-dimensional data alone. In this embodiment of the invention, the method comprises the following steps:
A.) providing an impression device comprising or consisting of an impression element which contains an impression material and which can be placed on a tooth or a tooth stump in order to take an impression of a tooth or a tooth stump;
B.) placing the impression device on a tooth or a tooth stump in an oral cavity, whereby an analogue impression of the tooth or tooth stump is produced in the impression material;
C.) removing the impression device from the oral cavity;
D.) before step B.) or after step C.), scanning the oral cavity with an intraoral scanner and producing a first data set which comprises a digital image of the oral cavity without the inserted impression device;
E.) scanning the analogue impression and producing a second data set which comprises a digitized three-dimensional image of the analogue impression; and
F.) registration of the first and second data set, in which a portion of the digital image of the tooth or the tooth stump in the first data set is brought into alignment with a portion of the digital image of the analogue impression in the second data set.

In contrast to the embodiment described above, in this alternative embodiment, the intraoral scan is carried out without an inserted impression device. Instead, the tooth or the tooth stump is scanned directly by means of an intraoral scan. As mentioned above, in this way the problem arises that the sub-gingival structures cannot be captured. For this purpose, however, an analogue impression of the tooth or the tooth stump is made in addition in the manner described above, which then is scanned in the step E.) defined above, whereupon a second data set is produced which captures the sub-gingival structures via the analogue impression. In this embodiment, both data sets are brought into registration such that in step F.) a portion of the digital image of the tooth or the tooth stump is brought into alignment with a portion of the digital image of the analogue impression in the second data set. A so-called best fit alignment is suitable for this purpose. A best fit alignment is an established method for bringing images into agreement. Broadly speaking, it is a minimization method, in which the sums of the distances between corresponding points on the objects to be brought into alignment are minimized.

The basic concept of combining an oral cavity scan and a scan of an analogue impression is also employed in this embodiment. The difference to the embodiment described above lies in the fact that the registration is carried out directly on the structures of the data sets instead of indirectly via corresponding markers in the two data sets.

Preferably, alignment of the first and second data sets comprises a best fit alignment.

### BRIEF DESCRIPTION OF THE FIGURES

Further details of the invention will now be given with reference to the accompanying drawings, in which:

FIG. 1 shows a cross-sectional view of a first embodiment of the impression device of the invention;

FIG. 2 shows a top view of the impression device of FIG. 1;

FIG. 3 shows a cross-sectional view of a second embodiment of the impression device of the invention;

FIG. 4 shows a top view of the impression device of FIG. 3;

FIG. 5 shows a cross-sectional view of a third embodiment of the impression device of the invention;

FIG. 6 shows a top view of the impression device of FIG. 5;

FIG. 7 shows a cross-sectional view of a fourth embodiment of the impression device of the invention;

FIG. 8 shows a top view of the impression device of FIG. 7;

FIG. 9 shows a cross-sectional view of an extraoral scanning device in accordance with a first embodiment with an inserted impression device in accordance with FIGS. 1 and 2;

FIG. 10 shows a top view of the extraoral scanning device of FIG. 9;

FIG. 11 shows a side view of an impression element provided with a geometric referencing marker;

FIG. 12 shows a top view of the impression element of FIG. 11, i.e. with a view onto the impression; and

FIG. 13 shows a view as in FIG. 12, but with an optical marker instead of a geometrical marker.

### DESCRIPTION OF THE PREFERRED EMBODIMENTS

FIG. 1 shows a first embodiment of the impression device 1 of the invention with an impression element formed as an impression sleeve 2, which is filled with an impression material 8. The impression sleeve 2 has an open and a closed end as well as a marker 3 which is connected to the impression sleeve 2 via a connecting element 7, wherein the connection element 7 extends from the closed end of the impression sleeve 2. Furthermore, an impression 30 formed in the impression material 8 can be seen.

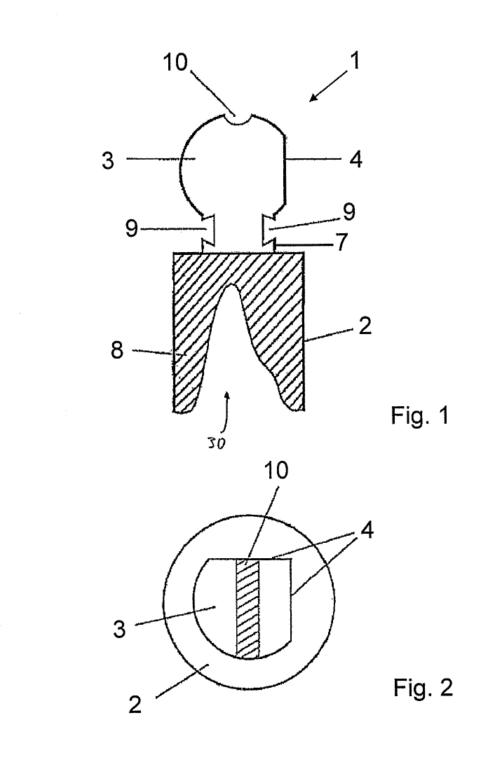

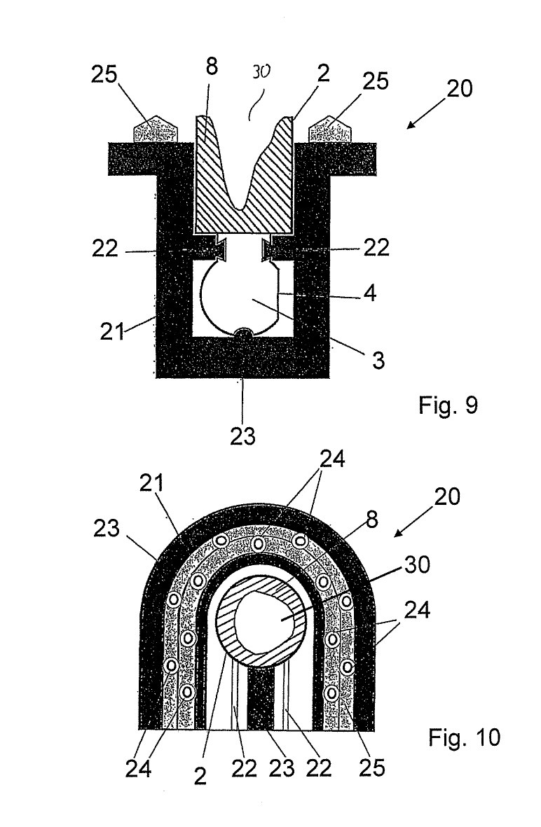

The marker 3 is at least partially spherical in shape and in the cross-sectional view shown, at least one flat 4 can be seen. As can be seen in FIG. 2, in addition to the flat 4 shown in FIG. 1, the marker 3 has a further flat 4, and the flats 4 are orientated at right angles to each other and each is of planar shape. The connecting element 7 has means 9 for spatially exact positioning of the impression device 1 in an extraoral scanning device 20 as shown in FIGS. 9 and 10. The positioning means 9 are formed as grooves of a dovetail connection and have a longitudinal axis which is orientated perpendicular to the plane of the drawing. The grooves 9 can engage with corresponding tongues which themselves are components of an extraoral scanning device 20 (see FIGS. 9 and 10) or a holder thereof, whereupon a tongue-and-groove connection is formed for spatially exact positioning of the impression device 1 in the extraoral scanning device 20.

The illustrated embodiment does not have a referencing marker, meaning that the referencing marker has to be provided by an extraoral scanning device 20. On the basis of the known position and orientation of the marker 3 with respect to the impression sleeve 2, the exact position and orientation of the impression element 1 in the oral cavity can be determined from the first data set obtained during the intraoral scan. In the extraoral scan, moreover, the shape and position and orientation of the impressed tooth or tooth stump with respect to the impression device 1 is determined from the second data set obtained by the extraoral scan taking the referencing marker into account. Finally, after registration of both data sets, in addition to the exact shape of the tooth or tooth stump including the sub-gingival structures, its exact position and orientation in the oral cavity can also be determined. In particular, the flat 4 on the otherwise spherical marker 3 can serve to determine the orientation of the tooth or tooth stump.

Furthermore, FIG. 2 more clearly shows the further means for spatially exact positioning of the impression device 1 in an extraoral scanning device 20; it is provided as a guide groove 10, which is formed on an end of the marker 3 distal to the impression sleeve 2.

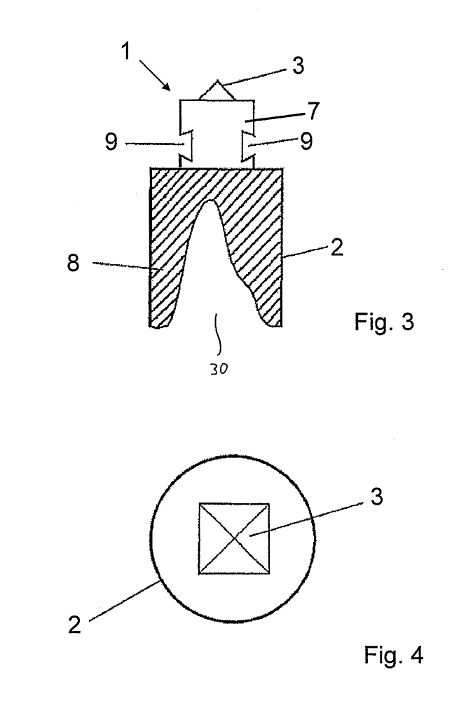

The embodiment of an impression device 1 of the invention shown in FIGS. 3 and 4 differs from that shown in FIGS. 1 and 2 in that the marker 3 is in the form of a pyramid with a square base. The pyramid is again connected with the impression sleeve 2 via a connecting element 7, wherein the connecting element 7 extends from the closed end of the impression sleeve 2 and comprises the positioning means 9 described for the previous embodiment.

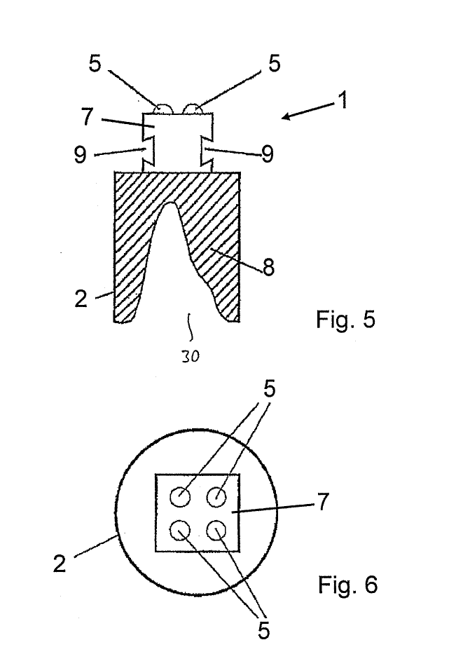

The embodiment of an impression device 1 shown in FIGS. 5 and 6 has four hemispherical elements 5 as the marker 3, which elements are arranged at the corners of a square.

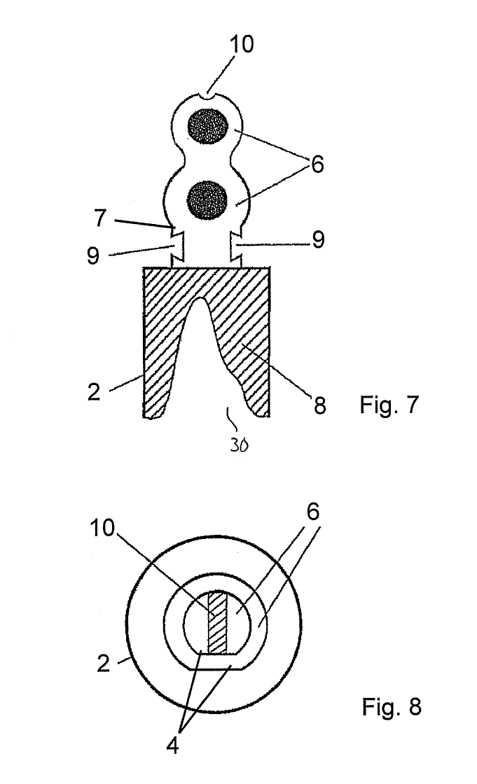

The marker 3 of the embodiment of FIGS. 7 and 8 comprises two spheres 6 arranged one above the other, wherein each of the spheres 6 has exactly one flat 4, which are arranged parallel to each other. The spheres 6 of the marker 3 which are arranged one above the other are again connected with the closed end of the impression sleeve 2 via a connecting element 7 adjacent to the lower sphere 6. As in the previous embodiments, the connecting element 7 comprises means 9 for spatially exact positioning of the impression device 1 in an extraoral scanning device 20 in accordance with FIGS. 9 and 10, wherein the positioning means 9 are formed as grooves of a dovetail connection. The marker 3 comprises further means for spatially exact positioning which is formed as a guide groove 10 formed on the end of the marker 3 distal to the impression sleeve 2 and which can be engaged with a guide rib 23 (see FIGS. 9 and 10) provided by the extraoral scanning device 20 or a holder thereof.

FIGS. 9 and 10 show a cross-sectional view and a top view, respectively, of an extraoral scanning device 20, into which an impression device 1 in accordance with the embodiment of FIGS. 1 and 2 has been inserted. Herein, the impression device 1 and the extraoral scanning device 20 in combination form a system for three-dimensional acquisition of intraoral structures. The extraoral scanning device 20 comprises a holder 21 in which the impression device 1 is accommodated. Like the impression device 1, the holder 21 comprises means 22 for spatially exact positioning of the impression device 1 with respect to the holder 21 and a referencing marker 24. In the embodiment shown, the positioning means 9 (see also FIG. 1) of the impression device 1 cooperate with the means 22 of the holder 21 for spatially exact positioning of the impression device 1. They form a dovetail connection. Finally, the holder 21 comprises a guide rib 23, which engages in a guide groove 10 (see also FIG. 1), which is formed on the end of the marker distal to the impression sleeve 2. The referencing markers 24 are provided on a U-shaped reference ring 25.

The longitudinal axes of the means 22, the positioning means 9, the guide groove 10 and the guide rib 23 are parallel to each other. This means that the impression device 1 for spatially exact positioning in the scanning device 20 can be pushed linearly into the scanning device 20 or the holder 21. The described means thus define the position of the impression device 1 in the scanning device 20 up to the degree of freedom in the insertion direction. The exact position in the insertion direction can be set by a stop, a catching position or the like.

After scanning both the impression that has been taken and the referencing marker 24 provided on the scanning device 20, knowing the disposition of the referencing marker 24 and the impression device 1 with respect to each other, and using the information obtained from the intraoral scan regarding the position of the impression device 1 when taking the impression in the oral cavity, the exact position, orientation and geometry of the tooth or tooth stump in the oral cavity can be determined.

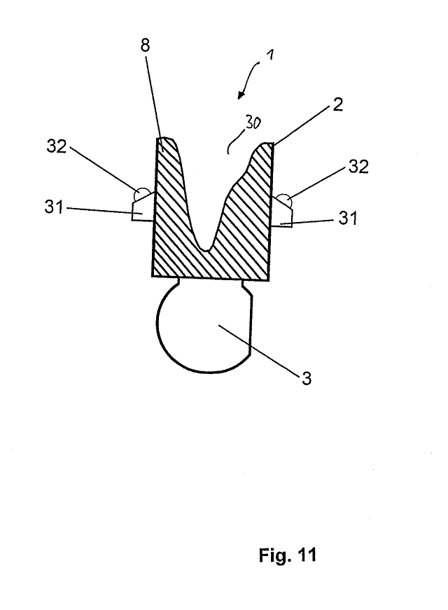

As mentioned above, it is not necessary for the referencing marker 24 to be provided on the scanning device 20. Instead, it is also possible to provide a referencing marker directly on the impression device 1 which reference marker is suitable for acquisition in the extraoral scan at the same time. Examples thereof are shown in FIGS. 11, 12 and 13.

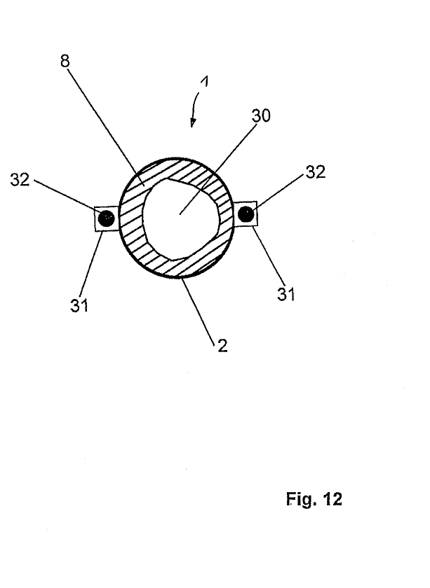

FIG. 11 shows a cross-sectional view of an impression device 1 which is basically similar in construction to those shown in the embodiments described above. In addition to the marker 3, however, this impression device 1 contains a marker arrangement 31 which in the embodiment shown carries a spherical referencing marker 32. Since the three-dimensional shape of the referencing marker 32 is distinctive, it is also termed a “geometrical marker”. The referencing marker 32 is disposed such that it can be accurately captured along with the impression 30 in the impression material 8 during the extraoral scan. This can be seen in particular in FIG. 12, which shows a top view of the image of FIG. 11, i.e. looking into the impression 30 in the sleeve 2. As can be seen in FIG. 12, in this view the marker arrangement 31 with the referencing marker 32 can clearly be seen.

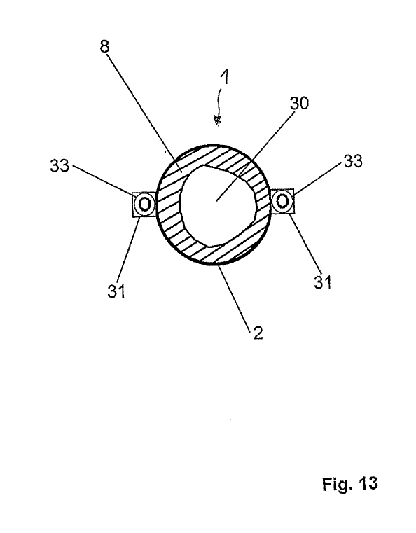

FIG. 13 shows an alternative embodiment in which the marker arrangement 31 is not geometric but a so-called “optical marker” 33. An “optical” marker is characterized by a specific optical configuration, in this embodiment shown as a dark sleeve on a light circle, rather than a specific geometry or three-dimensional shape. The referencing markers 32 and 33 are given purely by way of example.

A preferred embodiment of the invention is thus based on carrying out complete acquisition of the jaw, the associated teeth and the gingivae with the aid of a digital 3D acquisition system. To this end, in a first step the physician will take an analogue impression of the prepared teeth using an impression sleeve. The individual steps are as follows:
a) preparation of teeth;
b) selecting the appropriate size for the respective impression sleeve, which in each case can be selected having regard to the size and geometry of the prepared tooth. An individual adaptation of the impression sleeve by partially shortening it may be considered. The impression sleeve may either be produced from a polymer or from a metal. A particularly suitable polymer is PEEK (polyether ether ketone), since this material lends itself well to scanning. Metallic impression sleeves formed from titanium have proved to be particularly suitable. Preferably, such impression sleeves are provided with a coating which improves the suitability of the sleeves for scanning.

The impression sleeve of a preferred embodiment of the invention has a characteristic geometry. It comprises, for example, an essentially hollow cylindrical construction, wherein the base is preferably circular or oval. Ideally, the base is selected to suit the cross section of the various basic tooth types. Thus, the reference cross section should be the horizontal section through the appropriate tooth type at the transition between the root and the crown of the tooth. Thus, each tooth type may require a different size and length of sleeve.

The geometry of the marker may be particularly selected such that it can be acquired using routine intraoral scanners as geometrically accurately as possible.

When taking an analogue impression, the impression sleeve is filled with impression material and then placed onto the prepared tooth or tooth stump. Any superfluous material that might overflow out of the sides can be removed. The impressions of the teeth or tooth stumps may be taken either individually or simultaneously. For intraoral scanning, the impression sleeves remain in situ.

In a next step, digital 3D acquisition is carried out using an intraoral scanner. To this end, all of the structures required for further processing are acquired, in particular teeth, gingivae, prepared tooth stumps and teeth in the opposing jaw. The acquisition of sub-gingival regions of the prepared teeth or tooth stumps is not possible in this case. However, these already have been acquired by the analogue impression. The scannable impression sleeve or sleeves with its marker is also digitized in this step.

For extraoral scanning using the extraoral scanning device, after curing the impression material, the impression device is withdrawn from the prepared tooth or tooth stump. Next, the impression device is positioned in the extraoral scanning device. This can, for example, be accomplished by pushing it in laterally, whereupon a tongue-and-groove connection is formed between the components of the means for spatially exact positioning of the impression device in the extraoral scanning device of the impression device and also of the extraoral scanning device. This can, for example, be formed as a dovetail connection or a V-shaped guide. Preferably, such connections are formed on opposite sides of the connecting element of the impression device between the impression device and the extraoral scanning device or a holder thereof. In this manner, the impression device can be connected with the extraoral scanning device by pushing it in. Herein, the end position in the push direction can, for example, be set by means of a stop.

In a subsequent step, the tooth or tooth stump impression in the impression sleeve is preferably scanned with optical light, either with the aid of a separate scanner or with the aid of the digital 3D intraoral scanner already used for the intraoral scanning procedure.

By using a referencing marker which is acquired during the extraoral scan of the impression device, the exact position of the tooth stump modelled with the impression device in the complete image of the jaw, which has been acquired with the aid of the intraoral scanner, can be determined. The result is a digital intraoral impression which, in addition to the scan of the whole jaw, also comprises scan data concerning the sub-gingival preparation.

In an alternative embodiment, the marker 3 of the impression device 1 and the referencing markers 24, 32 or 33 can be dispensed with. Instead, the intraoral scan of the oral cavity is taken without an impression device 1, in order to produce a first data set which comprises a digital image of the oral cavity. Before or afterwards (not, however, during the intraoral scan), an impression device 1 is placed on a tooth or a tooth stump within the oral cavity, whereupon an analogue impression 30 of the tooth or the tooth stump is produced in the impression material 8. The impression device 1 may in fact be one of the types described above, with the exception that no marker 3 is required.

The analogue impression 30 in the impression device 1 is then scanned extraorally in order to obtain a second data set. This second data set contains no referencing markers 24, 32 or 33, in contrast to the embodiment described above. In fact, as before, this second data set contains all of the information regarding the analogue impression 30, i.e. even the sub-gingival structures, which were not imaged in the first data set for the reasons discussed above.

Finally, in this embodiment, the first and the second data sets are brought into registration by bringing a section of the digital image of the tooth or the tooth stump in the first data set into alignment with a section of the digital image of the analogue impression in the second data set, for example by means of a best fit alignment.

A particular feature in both of the embodiments described thus lies in the combination of analogue and digital impression techniques, which avoids the respective disadvantages and exploits the advantages of both impression techniques. Thus, it is possible to capture regions of the tooth digitally which otherwise could not be obtained by digital impression techniques. In particular, these are the sub-gingival regions of prepared teeth or tooth stumps.

The features of the invention disclosed in the present description, drawings and claims may be essential both individually or in any combination in implementing the invention.

### LIST OF REFERENCE NUMERALS

1 Impression device
2 Impression sleeve
3 Marker
4 Flat
5 Hemispherical element
6 Sphere
7 Connecting element
8 Impression material
9 Means for spatially exact positioning of impression device
10 Guide groove
20 Scanning device
21 Holder
22 Means for spatially exact positioning of impression device
23 Guide rib
24 Referencing marker
25 Reference ring
30 Impression
31 Marker arrangement
32 Geometrical referencing marker
33 Optical referencing marker

1. An impression device for three-dimensional acquisition of intraoral structures, having an impression element which can be placed on a tooth or a tooth stump in order to take an impression of the tooth or tooth stump, and at least one marker which is disposed on the impression element in a manner such that the position and the orientation of the impression element placed on the tooth or on the tooth stump can be determined by means of an intraoral scan, wherein the impression device comprises at least one referencing marker or can be connected to at least one referencing marker which is arranged such that it can be scanned together with the impression in an extraoral scan.

2. The impression device as claimed in claim 1, wherein the impression element comprises a sleeve to accommodate an impression material, having an open and a closed end, wherein the marker is disposed at the closed end.

3. The impression device as claimed in claim 1, wherein the marker has one or more of the characteristics selected from a group consisting of an at least partially spherical or pyramidal in shape, an at least partially planar lateral flat, a hemispherical element, and two spheres arranged one above the other, wherein at least one of the spheres has at least one flat.

4. The impression device as claimed in claim 2, wherein the marker is connected with the impression sleeve via a connecting element, wherein the connecting element extends from the closed end of the impression sleeve.

5. The impression device as claimed in claim 4, wherein the connecting element comprises positioning means for spatially exact positioning of the impression device in an extraoral scanning device.

6. The impression device as claimed in claim 5, wherein the positioning means have at least one groove or a tongue of a tongue-and-groove connection.

7. The impression device as claimed in claim 1, wherein the marker has a positioning means for spatially exact positioning of the impression device in an extraoral scanning device.

8. The impression device as claimed in claim 7, wherein the positioning means of the marker comprises a guide groove or a guide rib, which is formed on an end of the marker which is distal to the impression sleeve.

9. A system for three-dimensional acquisition of intraoral structures, said system comprising:
an impression device for three-dimensional acquisition of intraoral structures, having an impression element which can be placed on a tooth or a tooth stump in order to take an impression of the tooth or tooth stump, and at least one marker which is disposed on the impression element in a manner such that the position and the orientation of the impression element placed on the tooth or on the tooth stump can be determined by means of an intraoral scan, and
an extraoral scanning device for extraoral scanning of an impression of a tooth or a tooth stump recorded with the aid of said impression device, wherein the scanning device comprises positioning means for spatially exact positioning of the impression device with respect to the scanning device.

10. The system as claimed in claim 9, wherein the extraoral scanning device comprises at least one referencing marker which is arranged such that it can be scanned together with the impression in an extraoral scan, when the impression device is accommodated in the extraoral scanning device.

11. The system as claimed in claim 9, wherein the impression device comprises positioning means cooperating with the positioning means of the scanning device for spatially exact positioning of the impression device.

12. The system as claimed in claim 11, wherein the cooperating positioning means form a tongue-and-groove connection.

13. The system as claimed in claim 9, wherein the impression element comprises a sleeve to accommodate an impression material and the external scanning device comprises a guide rib, which can be engaged with a guide groove formed on the end of the marker distal to said impression sleeve.

14. A method for three-dimensional acquisition of intraoral structures, said method comprising the following steps:
providing an impression device, said impression device comprising a marker and an impression element filled with an impression material;
placing the impression device on a tooth or a tooth stump in an oral cavity, such that an analogue impression of the tooth or the tooth stump is produced in the impression material;
scanning the oral cavity including the marker disposed on the impression sleeve with an intraoral scanner and producing a first data set which comprises a digitized, three-dimensional image of the oral cavity and the marker;
removing the impression device from the oral cavity and scanning the analogue impression as well as the referencing marker and producing a second data set which comprises a digitized three-dimensional image of the analogue impression and the referencing marker.

15. A method as claimed in claim 14, wherein for scanning, the impression device is inserted into an extraoral scanning device, said extraoral scanning device comprising positioning means for spatially exact positioning of the impression device with respect to the extraoral scanning device.

16. A method as claimed in claim 14, wherein the first and the second data sets are brought into registration based on a known spatial relationship between the at least one marker of the impression device and the referencing marker.

17. A method for three-dimensional acquisition of intraoral structures, having the following steps:
A.) providing an impression device comprising or consisting of an impression element which contains an impression material and which can be placed on a tooth or a tooth stump in order to take an impression of a tooth or a tooth stump;
B.) placing the impression device on a tooth or a tooth stump in an oral cavity, whereby an analogue impression of the tooth or tooth stump is produced in the impression material;
C.) removing the impression device from the oral cavity;
D.) before step B.) or after step C.), scanning the oral cavity with an intraoral scanner and producing a first data set which comprises a digital image of the oral cavity without the inserted impression device;
E.) scanning the analogue impression and producing a second data set which comprises a digitized three-dimensional image of the analogue impression; and
F.) registration of the first and second data set, in which a portion of the digital image of the tooth or the tooth stump in the first data set is brought into alignment with a portion of the digital image of the analogue impression in the second data set.

18. A method as claimed in claim 17, wherein the section of the digital image of the tooth or the tooth stump from the first data set is associated with the digital image of the analogue impression from the second data set by means of a best fit alignment.
